(19) United States
(12) Patent Application Publication
(10) Pub. No.: US 2013/0046122 A1
(43) Pub. Date: Feb. 21, 2013

(54) DEBOTTLENECKING OF A STEAM CRACKER UNIT TO ENHANCE PROPYLENE PRODUCTION

(71) Applicants: Walter Vermeiren, Houthalen (BE); François Bouvart, Senlis (FR); Ineke Celie, De Pinte (BE); Wolfgang Garcia, Braine-l'Alleud (BE)

(72) Inventors: Walter Vermeiren, Houthalen (BE); François Bouvart, Senlis (FR); Ineke Celie, De Pinte (BE); Wolfgang Garcia, Braine-l'Alleud (BE)

(73) Assignee: TOTAL PETROCHEMICALS RESEARCH FELUY, Seneffe (Feluy) (BE)

(21) Appl. No.: 13/513,892

(22) PCT Filed: Dec. 15, 2010
(86) PCT No.: PCT/EP2010/069694

§ 371 Date: Oct. 1, 2012

(30) Foreign Application Priority Data
Dec. 15, 2009 (EP) 09179240.8

(51) Int. Cl. C07C 5/02

(52) U.S. Cl. 585/251

(57) ABSTRACT

The present invention is method for debottlenecking an existing steam cracker unit of which the operation is modified from high severity to low severity operation, having a cracking zone and a fractionation zone, said fractionation zone comprising a gasoline stripper, a de-methaniser (I), a de-ethaniser (I) a de-propaniser (I) and a de-butaniser (I), said de-propaniser (I) receiving product from the bottom of the de-ethaniser (I) and optionally product from the bottom of the gasoline stripper (I), wherein said debottlenecking method comprises the steps of:
a) adding a selective hydrogenation unit (II),
b) adding a cracking reactor (II) comprising a catalyst selective towards light olefins in the outlet,
c) adding a re-run column and a de-propaniser (II),
d) sending a part or all of the bottoms stream of the gasoline stripper (I) to the selective hydrogenation unit (II) and subsequently to the cracking reactor (II) at conditions effective to produce an outlet with an olefin content of lower molecular weight than that of the inlet,
e) sending a part of the bottoms stream of the de-ethaniser (I) to the de-propaniser (II), such as, not to overload the de-propaniser (I)
f) optionally sending a part or all of the overhead raw C₄ fraction of the de-butaniser (I) to the selective hydrogenation unit (II),
g) sending the cracking reactor (II) outlet to the re-run column to produce a C₆+ bottom stream and a C₁-C₅ overhead, sending said overhead to the de-propaniser (II) to produce a C₁-C₃ overhead and a C₄+ bottom stream recycled in whole or in part to the selective hydrogenation unit (II), optionally withdrawing a part of said C₄+ bottom stream.

### FIELD OF THE INVENTION

The present invention relates to the debottlenecking or change in operating conditions of a steam cracker unit in order to enhance propylene production.

Steam cracking of hydrocarbons (also referred as thermal cracking or pyrolysis) is a non-catalytic petrochemical process that is widely used to produce olefins such as ethylene, propylene, butenes, butadiene, and aromatics such as benzene, toluene, and xylenes. Basically, a hydrocarbon feedstock such as naphtha, gas oil or other fractions of whole crude oil that are produced by distilling or otherwise fractionating whole crude oil, is mixed with steam which serves as a diluent to keep the partial pressure of hydrocarbon molecules low. The steam/hydrocarbon mixture is preheated to from about 480° C. to about 540° C., and then enters the reaction zone where it is very quickly heated to an hydrocarbon thermal cracking temperature. Thermal cracking is accomplished without the aid of any catalyst. This process is carried out in a pyrolysis furnace (steam cracker) at pressures in the reaction zone ranging from about 10 to about 30 psig. Pyrolysis furnaces have internally thereof a convection section and a radiant section. Preheating is accomplished in the convection section, while cracking occurs in the radiant section.

After the thermal cracking, the effluent from the pyrolysis furnace (the cracking zone) contains gaseous hydrocarbons of great variety, e.g., from one to thirty-five carbon atoms per molecule. These gaseous hydrocarbons can be saturated, monounsaturated, and polyunsaturated, and can be aliphatic, alicyclics, and/or aromatic. The cracked gas also contains significant amounts of molecular hydrogen (hydrogen). The cracked product is then further processed in a fractionation section to produce, as products of the plant, various separate individual streams of high purity such as hydrogen, ethylene, propylene, mixed hydrocarbons having four carbon atoms per molecule, fuel oil, and pyrolysis gasoline. Each separate individual stream aforesaid is a valuable commercial product.

The proportions of the various products obtained depend significantly upon cracking severity, which can be expressed in terms of methane yield since methane is the ultimate hydrocarbon product. At a low severity, i.e. at methane yields below about 4 or 6 weight percent based on feed oil, yields of most products will be low. At a moderate severity, i.e. at methane yields above about 4 or 6 but below about 12 or 14 weight percent, optimum yields of intermediate olefins such as propylene and 1,3-butadiene will be realized. At high severities, i.e. at methane yields above about 12 or 14 weight percent, yields of propylene and 1,3-butadiene will decline and yields of very light materials, such as methane, hydrogen, and ethylene will tend to increase. Severity is increasing with the temperature increase of the cracking zone as well with the decrease of the residence time in said cracking zone.

In the past steam crackers were typically designed for maximum ethylene production and operated hence at high severity, resulting in high methane and ethylene production and relative lower propylene production. Such a steam cracker is hence designed for maximum fractionation capacity of lights ends (hydrogen and methane) and ethylene in the de-methaniser and de-ethaniser, respectively. When there is a desire to reduce the severity of a steam cracker for the same naphtha flow rate, less methane and ethylene will be produced on one hand and more propylene and C₄+ will be produced on the other hand. The sum of ethylene and propylene are nearly the same. However, when changing to low severity operation often the propylene section (de-propaniser and C₃ splitter) and the C₄+ section (de-butaniser, de-pentaniser etc) become bottlenecked. Moreover, when operating a steamcracker at lower severity, the furnace duty per ton of feed and the pressure drop over the furnace tubes decreases as a smaller amount of lighter molecules are produced. This allows increasing the naphtha throughput up to a similar furnace duty and pressure drop as for the high severity operation. This allows filling the fractionation capacity of the lights ends (hydrogen and methane) and ethylene in the de-methaniser and de-ethaniser, respectively, again to their maximum design values. However, the bottleneck in the fractionation of heavier molecules (propylene and C₄+ ) will become even worse. The revamping of the above steam cracker to enhance the propylene production needs to operate at a lower severity but in the fractionation zone the C₂ section becomes optionally oversized and the C₃-C₄ section undersized. Often for existing steamcrackers most sections have already been debottlenecked several times by optimising reboiling and condensing duties and installing more efficient trays or packings. This implies that when changing the operation to lower severity, major modification like new reboilers, condensers or even full distillation columns need to be installed. The present invention concerns said revamping,

### BACKGROUND OF THE INVENTION

WO 03-099964 describes a process for steam cracking a hydrocarbon feedstock containing olefins to provide increased light olefins in the steam cracked effluent, the process comprising passing a first hydrocarbon feedstock containing one or more olefins through a reactor containing a crystalline silicate to produce an intermediate effluent with an olefin content of lower molecular weight than that of the feedstock, fractionating the intermediate effluent to provide a lower carbon fraction and a higher carbon fraction, and passing the higher carbon fraction, as a second hydrocarbon feedstock, through a steam cracker to produce a steam cracked effluent.

US 2003-220530 describes a process for preparing olefins from a hydrocarbon-containing feed, comprising:
introducing hydrocarbon-containing feed to a treatment plant wherein at least one hydrocarbon-containing fraction at least part comprising relatively long-chain olefins is produced;
feeding same hydrocarbon-containing fraction to an olefin conversion stage in which at least part of the relatively long-chain olefins is converted into relatively shorter-chain olefins; and
recirculating at least part of said relatively shorter-chain olefins to the treatment plant; wherein said olefin conversion stage is preceded by a paraffin/olefin separation stage, in which olefins and paraffins are separated from one another, at least part of the separated paraffins are fed to the treatment plant or taken off and passed to another use, and at least part of the separated olefins are fed to the olefin conversion stage.

US 2007-100182 concerns a process for producing propylene and co-producing desulphurized gasoline with a high octane number from a catalytically cracked gasoline cut, and a steam cracking C4/C5 cut comprising at least one one-step oligocracking unit, a selective hydrogenation unit for FCC gasoline and a hydrotreatment unit.

These prior arts don't concern the debottlenecking of a steam cracker.

### BRIEF SUMMARY OF THE INVENTION

The present invention is method for debottlenecking an existing steam cracker unit of which the operation is modified from high severity to low severity operation, having a cracking zone and a fractionation zone, said fractionation zone comprising a gasoline stripper, a de-methaniser (I), a de-ethaniser (I) a de-propaniser (I) and a de-butaniser (I), said de-propaniser (I) receiving product from the bottom of the de-ethaniser (I) and optionally product from the bottom of the gasoline stripper (I), wherein said debottlenecking method comprises the steps of:
a) adding a selective hydrogenation unit (II),
b) adding a cracking reactor (II) comprising a catalyst selective towards light olefins in the outlet,
c) adding a re-run column and a de-propaniser (II),
d) sending a part or all of the bottoms stream of the gasoline stripper (I) to the selective hydrogenation unit (II) and subsequently to the cracking reactor (II) at conditions effective to produce an outlet with an olefin content of lower molecular weight than that of the inlet,
e) sending a part of the bottoms stream of the de-ethaniser (I) to the de-propaniser (II), such as, not to overload the de-propaniser (I)
f) optionally sending a part or all of the overhead raw C₄ fraction of the de-butaniser (I) to the selective hydrogenation unit (II),
g) sending the cracking reactor (II) outlet to the re-run column to produce a C₆+ bottom stream and a C₁-C₅ overhead, sending said overhead to the de-propaniser (II) to produce a C₁-C₃ overhead and a C₄+ bottom stream recycled in whole or in part to the selective hydrogenation unit (II), optionally withdrawing a part of said C₄+ bottom stream.

More precisely the present invention is a method for debottlenecking an existing steam cracker unit of which the operation is modified from high severity to low severity operation, having a cracking zone and a fractionation zone, said fractionation zone comprising a gasoline stripper, a de-methaniser (I), a de-ethaniser (I) a de-propaniser (I) and a de-butaniser (I), in which, said de-ethaniser (I) is producing,
an overhead stream comprising ethylene, ethane and optionally fuel gas,
a bottoms stream comprising C₃+ sent to the de-propaniser (I),
   said de-propaniser (I) receiving product from the bottom of the de-ethaniser (I) and optionally product from the bottom of the gasoline stripper (I), said de-propaniser (I) producing,
an overhead stream sent to a MAPD removing unit (I) to produce propane and propylene,
a bottoms stream comprising C₄+ sent to the de-butaniser (I) to produce an overhead raw C₄ fraction and a bottom C₅+ fraction,
   wherein said debottlenecking method comprises the steps of:
a) adding a selective hydrogenation unit (II),
b) adding a cracking reactor (II) comprising a catalyst selective towards light olefins in the outlet,
c) adding a re-run column and a de-propaniser (II),
d) sending a part or all of the bottoms stream of the gasoline stripper (I) to the selective hydrogenation unit (II) and subsequently to the cracking reactor (II) at conditions effective to produce an outlet with an olefin content of lower molecular weight than that of the inlet,
e) sending a part of the bottoms stream of the de-ethaniser (I) to the de-propaniser (II), such as, not to overload the de-propaniser (I)
f) optionally sending a part or all of the overhead raw C₄ fraction of the de-butaniser (I) to the selective hydrogenation unit (II),
g) sending the cracking reactor (II) outlet to the re-run column to produce a C₆+ bottom stream and a C₁-C₅ overhead, sending said overhead to the de-propaniser an to produce a C₁-C₃ overhead and a C₄+ bottom stream recycled in whole or in part to the selective hydrogenation unit (II), optionally withdrawing a part of said C₄+ bottom stream.

In a first embodiment (front-end de-methaniser) the present invention is a method for debottlenecking an existing steam cracker unit of which the operation is modified from high severity to low severity operation, having a cracking zone and a fractionation zone, said fractionation zone comprising a gasoline stripper, then a fractionation configuration with first a de-methaniser (I) (front-end de-methaniser), followed by a de-ethaniser (I) and followed by a de-propaniser (I) and a de-butaniser (I), in which, said de-ethaniser (I) is producing,
an overhead stream sent to a C₂ splitter (I) through a back-end acetylene converter (I) to separate ethylene and ethane,
a bottoms stream comprising C₃+ sent to the de-propaniser (I),
   said de-propaniser (I) receiving product from the bottom of the de-ethaniser (I) and optionally product from the bottom of the gasoline stripper (I), said de-propaniser (I) producing,
an overhead stream sent to a MAPD removing unit (I) to produce propane and propylene,
a bottoms stream comprising C₄+ sent to the de-butaniser (I) to produce an overhead raw C₄ fraction and a bottom C₅+ fraction, optionally said C₅+ fraction is subsequently sent to a de-pentaniser (I) to produce an overhead C₅ fraction and a bottom C₆+ fraction,
   wherein said debottlenecking method comprises the steps of:
a) adding a selective hydrogenation unit (II),
b) adding a cracking reactor (II) comprising a catalyst selective towards light olefins in the outlet,
c) adding a re-run column, a de-propaniser (II), a de-ethaniser (II), optionally a de-methaniser (II), optionally a MAPD conversion unit (II) and optionally a C₃ splitter (II) to separate propane and propylene,
d) sending a part or all of the bottoms stream of the gasoline stripper (I) to the selective hydrogenation unit (II) and subsequently to the cracking reactor (II) at conditions effective to produce an outlet with an olefin content of lower molecular weight than that of the inlet,
e) sending a part of the bottoms stream of the de-ethaniser (I) to the de-propaniser (II), such as, not to overload the de-propaniser (I)
f) optionally sending a part or all of the overhead raw C₄ fraction of the de-butaniser (I) or a part or all of the overhead C₅ fraction of the de-pentaniser (I) or imported olefinic C₄+ hydrocarbons or any mixture of the above to the selective hydrogenation unit (II),
g) sending the cracking reactor (II) outlet to the re-run column to produce a C₆+ bottom stream and a C₁-C₅ overhead, sending said overhead to the de-propaniser (II) to produce a C₁-C₃ overhead and a C₄+ bottom stream recycled in whole or in part to the selective hydrogenation unit (II), optionally withdrawing a part of said C₄+ bottom stream,
h) sending the C₁-C₃ overhead of the de-propaniser (II) to the de-ethaniser (II) to produce
   a bottom C₃ stream optionally sent to the MAPD converter (II) to produce propane and propylene stream, optionally sent to the C₃ splitter (II) to produce a concentrated propylene stream as overhead and a propane rich bottom product,
   an overhead stream optionally sent to a de-methaniser (II) to produce an overhead fuel gas and a C₂ bottom stream optionally sent to an acetylene converter.

In a second embodiment (front-end de-ethaniser) the present invention is a method for debottlenecking an existing steam cracker unit of which the operation is modified from high severity to low severity operation, having a cracking zone and a fractionation zone, said fractionation zone comprising a gasoline stripper, then a fractionation configuration with first a de-ethaniser (I) (front-end de-ethaniser), followed by a de-methaniser (I) and followed by a de-propaniser (I) and a de-butaniser (I), in which, said de-ethaniser (I) is producing,
an overhead stream sent to a front-end acetylene converter (I) and then to a de-methaniser (I), said de-methaniser (I) producing a fuel gas overhead product and a C₂ bottom product that is sent to a C₂ splitter (I) to separate ethylene and ethane,
a bottoms stream comprising C₃+ sent to the de-propaniser (I),
   said de-propaniser (I) receiving product from the bottom of the de-ethaniser (I) and optionally product from the bottom of the gasoline stripper (I), said de-propaniser (I) producing,
an overhead stream sent to a MAPD removing unit (I) to produce propane and propylene,
a bottoms stream comprising C₄+ sent to the de-butaniser (I) to produce an overhead raw C₄ fraction and a bottom C₅+ fraction, optionally said C₅+ fraction is subsequently sent to a de-pentaniser (I) to produce an overhead C₅ fraction and a bottom C₆+ fraction,
   wherein said debottlenecking method comprises the steps of:
a) adding a selective hydrogenation unit (II),
b) adding a cracking reactor (II) comprising a catalyst selective towards light olefins in the outlet,
c) adding a re-run column, a de-propaniser (II), a de-ethaniser (II), optionally a de-methaniser (II), optionally a MAPD conversion unit (II) and optionally a C₃ splitter (II) to separate propane and propylene,
d) sending a part or all of the bottoms stream of the gasoline stripper (I) to the selective hydrogenation unit (II) and subsequently to the cracking reactor (II) at conditions effective to produce an outlet with an olefin content of lower molecular weight than that of the inlet,
e) sending a part of the bottoms stream of the de-ethaniser (I) to the de-propaniser (II), such as not to overload the de-propaniser (I)
f) optionally sending a part or all of the overhead raw C₄ fraction of the de-butaniser (I) or a part or all of the overhead C₅ fraction of the de-pentaniser (I) or imported olefinic C₄+ hydrocarbons or any mixture of the above to the selective hydrogenation unit (II),
g) sending the cracking reactor (II) outlet to the re-run column to produce a C₆+ bottom stream and a C₁-C₅ overhead, sending said overhead to the de-propaniser (II) to produce a C₁-C₃ overhead and a C₄+ bottom stream recycled in whole or in part to the selective hydrogenation unit (II), optionally withdrawing a part of said C₄+ bottom stream,
h) sending the C₁-C₃ overhead of the de-propaniser (II) to the de-ethaniser (II) to produce
   a bottom C₃ stream optionally sent to the MAPD converter (II) to produce propane and propylene stream, optionally sent to the C₃ splitter (II) to produce a concentrated propylene stream as overhead and a propane rich bottom product,
   an overhead stream optionally sent to a de-methaniser (II) to produce an overhead fuel gas and a C₂ bottom stream optionally sent to an acetylene converter.

In a specific embodiment the MAPD removal unit (I) consists in a MAPD converter. This MADP converter can be a catalytic gas phase or liquid phase reactor that converts the MAPD (methyl acetylene and propadiene) selectively in mainly propylene. The MAPD converter can consist in a one-stage or a two-stage reactor with intermediate cooling and hydrogen addition.

In a specific embodiment the MAPD removal unit (I) consists in a MAPD distillation column fed with the overhead of the de-propaniser (I) and a MAPD converter. The MAPD distillation column produces a C₃ overhead product, having substantially C₃ hydrocarbons and less MAPD than in the feed to the column and a bottom product comprising higher concentration of MAPD and other C₄+ hydrocarbons (commonly called tetrene). The overhead of the MAPD distillation column is sent to a MAPD converter that converts the MAPD (methyl acetylene and propadiene) selectively in mainly propylene to produce a propane and propylene stream. The bottom product of the MAPD distillation column is optionally sent, in whole or in part, to the de-propaniser (II). The MAPD converter can be gas phase or a liquid stage catalytic converter.

In a specific embodiment the MAPD removal unit (I) consists in a catalytic MAPD distillation column (I) and optionally a MAPD converter (I). Said catalytic MAPD distillation column (I) is fed with the overhead of the de-propaniser (I) and comprises a selective hydrogenation catalyst placed inside a distillation column. In said catalytic MAPD distillation column (I) MAPD (methyl acetylene and propadiene) and at least a part of the C₄+ dienes and alkynes are substantially hydrogenated into the corresponding olefins. Advantageously in said catalytic MAPD distillation column (I) acetylenic and dienic hydrocarbons are selectively converted into the corresponding olefins. The overhead of said catalytic MAPD distillation column (I), having substantially C₃ hydrocarbons, is optionally sent to a finishing MAPD converter (I) to produce propane and propylene. In the finishing MAPD converter (I) the remaining MAPD is converted to propylene. The bottoms of said catalytic MAPD distillation column (I), having substantially C₄ hydrocarbons, are optionally sent, in whole or in part, to the de-propaniser (II) or optionally to the selective hydrogenation unit (II). The finishing MAPD converter (I) can be gas phase or a liquid stage catalytic converter.

In a specific embodiment the de-propaniser (I) is a catalytic de-propaniser (I). Said catalytic de-propaniser (I) fed with the bottom product of the de-ethaniser (I) and optionally product from the bottom of the gasoline stripper (I), and comprises a selective hydrogenation catalyst placed inside a distillation column and producing an overhead product, having substantially C₃ hydrocarbons and a bottom product, having substantially C₄+ hydrocarbons. In said catalytic de-propaniser (I) MAPD (methyl acetylene and propadiene) and at least a part of the C₄+ dienes and alkynes are substantially hydrogenated into the corresponding olefins. Advantageously in said catalytic de-propaniser (I) acetylenic and dienic hydrocarbons are selectively converted into the corresponding olefins. The C₃ overhead of said catalytic de-propaniser (I) is optionally sent to a finishing MAPD converter (I) to produce propane and propylene. The bottoms of said catalytic de-propaniser (I) are sent to the de-butaniser (I), optionally a part is sent to the de-propaniser (II) and/or to the selective hydrogenation unit (II). The finishing MAPD converter (I) can be gas phase or a liquid stage catalytic converter.

Advantageously the acetylene converter (I) is a two stages converter. Advantageously up to about 50 to 95% of the acetylene is converted in the first step. Advantageously in step h) the C₂ bottoms stream of the de-methaniser (II) is sent to the inlet of the first stage of the acetylene converter (I).

### DETAILED DESCRIPTION OF THE INVENTION

Steam cracking is a known process. Steamcrackers are complex industrial facilities that can be divided into three main zones, each of which has several types of equipment with very specific functions: (i) the hot zone including: pyrolysis or cracking furnaces, quench exchanger and quench ring, the columns of the hot separation train (ii) the compression zone including: a cracked gas compressor, purification and separation columns, dryers and (iii) the cold zone including: the cold box, de-methaniser, fractionating columns of the cold separation train, the C₂ and C₃ converters, the gasoline hydrostabilization reactor Hydrocarbon cracking is carried out in tubular reactors in direct-fired heaters (furnaces). Various tube sizes and configurations can be used, such as coiled tube, U-tube, or straight tube layouts. Tube diameters range from 1 to 4 inches. Each furnace consists of a convection zone in which the waste heat is recovered and a radiant zone in which pyrolysis takes place. The feedstock-steam mixture is preheated in the convection zone to about 530-650° C. or the feedstock is preheated in the convection section and subsequently mixed with dilution steam before it flows over to the radiant zone, where pyrolysis takes place at temperatures varying from 750 to 950° C. and residence times from 0.05 to 0.5 second, depending on the feedstock type and the cracking severity desired. The steam/feedstock weight ratio is between 0.2 and 1.0 kg/kg, preferentially between 0.3 and 0.5 kg/kg. For steamcracking furnaces, the severity can be modulated by: temperature, residence time, total pressure and partial pressure of hydrocarbons. In general the ethylene yield increases with the temperature while the yield of propylene decreases. At high temperatures, propylene is cracked and hence contributes to more ethylene yield. The increase in severity thus obtained leads to a moderate decrease in selectivity and a substantial decrease of the ratio C₃═/C₂═. So high severity operation favors ethylene, while low severity operation favors propylene production. The residence time of the feed in the coil and the temperature are to be considered together. Rate of coke formation will determine maximum acceptable severity. A lower operating pressure results in easier light olefins formation and reduced coke formation. The lowest pressure possible is accomplished by (i) maintaining the output pressure of the coils as close as possible to atmospheric pressure at the suction of the cracked gas compressor (ii) reducing the pressure of the hydrocarbons by dilution with steam (which has a substantial influence on slowing down coke formation). The steam/feed ratio must be maintained at a level sufficient to limit coke formation.

Effluent from the pyrolysis furnaces contains unreacted feedstock, desired olefins (mainly ethylene and propylene), hydrogen, methane, a mixture of C₄'s (primarily isobutylene and butadiene), pyrolysis gasoline (aromatics in the C₆ to C₈ range), ethane, propane, di-olefins (acetylene, methyl acetylene, propadiene), and heavier hydrocarbons that boil in the temperature range of fuel oil. This cracked gas is rapidly quenched to 338-510° C. to stop the pyrolysis reactions, minimize consecutive reactions and to recover the sensible heat in the gas by generating high-pressure steam in parallel transfer-line heat exchangers (TLE's). In gaseous feedstock based plants, the TLE-quenched gas stream flows forward to a direct water quench tower, where the gas is cooled further with recirculating cold water. In liquid feedstock based plants, a prefractionator precedes the water quench tower to condense and separate the fuel oil fraction from the cracked gas. In both types of plants, the major portions of the dilution steam and heavy gasoline in the cracked gas are condensed in the water quench tower at 35-40° C. The water-quench gas is subsequently compressed to about 25-35 Bars in 4 or 5 stages. Between compression stages, the condensed water and light gasoline are removed, and the cracked gas is washed with a caustic solution or with a regenerative amine solution, followed by a caustic solution, to remove acid gases (CO₂, H₂S and SO₂). The compressed cracked gas is dried with a desiccant and cooled with propylene and ethylene refrigerants to cryogenic temperatures for the subsequent product fractionation: Front-end demethanization, Front-end depropanization or Front-end deethanization.

In a front-end demethanization configuration, tail gases (CO, H₂, and CH₄) are separated from the C₂+ components first by de-methanization column at about 30 bars. The bottom product flows to the de-ethanization, of which the overhead product is treated in the acetylene hydrogenation unit and further fractionated in the C₂ splitting column. The bottom product of the de-ethanization goes to the de-propanization, of which the overhead product is treated in the methyl acetylene/propadiene hydrogenation unit and further fractionated in the C₃ splitting column. The bottom product of the de-propaniser goes to the de-butanization where the C₄'s are separated from the pyrolysis gasoline fraction. In this separation sequence, the H₂ required for hydrogenation is externally added to C₂ and C₃ streams. The required H₂ is typically recovered from the tail gas by methanation of the residual CO and optionally further concentrated in a pressure swing adsorption unit.

Front-end de-propanization configuration is used typically in steamcrackers based on gaseous feedstock. In this configuration, after removing the acid gases at the end of the third compression stage, the C₃ and lighter components are separated from the C₄+ by de-propanization. The de-propaniser C₃− overhead is compressed by a fourth stage to about 30-35 bars. The acetylenes and/or dienes in the C₃− cut are catalytically hydrogenated with H₂ still present in the stream. Following hydrogenation, the light gas stream is de-methanized, de-ethanized and C₂ split. The bottom product of the de-ethanization can optionally be C₃ split. In an alternative configuration, the C₃− overhead is first de-ethanised and the C₂− treated as described above while the C₃'s are treated in the C₃ acetylene/diene hydrogenation unit and C₃ split. The C₄+ de-propaniser bottom is de-butanized to separate C₄'s from pyrolysis gasoline.

There are two versions of the front-end de-ethanization separation sequence. The product separation sequence is identical to the front-end de-methanization and front-end depropanization separation sequence to the third compression stage. The gas is de-ethanized first at about 27 bars to separate C₂− components from C₃+ components. The overhead C₂− stream flows to a catalytic hydrogenation unit, where acetylene in the stream is selectively hydrogenated. The hydrogenated stream is chilled to cryogenic temperatures and de-methanized at low pressure of about 9-10 bars to strip off tail gases. The C₂ bottom stream is split to produce an overhead ethylene product and an ethane bottom stream for recycle. In parallel, the C₃+ bottom stream from the front-end de-ethaniser undergoes further product separation in a de-propaniser, of which the overhead product is treated in the methyl acetylene/propadiene hydrogenation unit and further fractionated in the C₃ splitting column. The bottom product of the de-propaniser goes to the de-butanization where the C₄'s are separated from the pyrolysis gasoline fraction. In the more recent version of the front-end de-ethanization separation configuration, the cracked gas is caustic washed after three compression stages, pre-chilled and is then de-ethanized at about 16-18 bars top pressure. The net overhead stream (C₂−) is compressed further in the next stage to about 35-37 bars before it passes to a catalytic converter to hydrogenate acetylene, with hydrogen still contained in the stream. Following hydrogenation, the stream is chilled and de-methanized to strip off the tail gases from the C₂ bottom stream. The C₂'s are split in a low pressure column operating at 9-10 bars pressure, instead of 19-24 bars customarily employed in high pressure C₂ splitters that use a propylene refrigerant to condense reflux for the column. For the low-pressure C₂ splitter separation scheme, the overhead cooling and compression system is integrated into a heat-pump, open-cycle ethylene refrigeration circuit. The ethylene product becomes a purged stream of the ethylene refrigeration recirculation system.

The ethane bottom product of the C₂ splitter is recycled back to steam cracking. Propane may also be re-cracked, depending on its market value. Recycle steam cracking is accomplished in two or more dedicated pyrolysis furnaces to assure that the plant continues operating while one of the recycle furnaces is being decoked.

Many other variations exist of the above-described configurations; in particular in the way the undesired acetylene/dienes are removed from the ethylene and propylene cuts.

As regards the cracking reactor (II), it is also known as OCP (Olefins Conversion Process) reactor and referred as an “OCP process”. Said reactor (II) contains any catalyst, referred as catalyst (A1), provided it is selective to light olefins. Said OCP process is known per se. It has been described in EP 1036133, EP 1035915, EP 1036134, EP 1036135, EP 1036136, EP 1036138, EP 1036137, EP 1036139, EP 1194502, EP 1190015, EP 1194500, EP 1363983 and WO 2009/016156 the content of which are incorporated in the present invention.

According to a first advantageous embodiment the catalyst (A1) is a crystalline silicate containing advantageously at least one 10 members ring into the structure. It is by way of example of the MFI (ZSM-5, silicalite-1, boralite C, TS-1), MEL (ZSM-11, silicalite-2, boralite D, TS-2, SSZ-46), FER (Ferrierite, FU-9, ZSM-35), MTT (ZSM-23), MWW (MCM-22, PSH-3, ITQ-1, MCM-49), TON (ZSM-22, Theta-1, NU-10), EUO (ZSM-50, EU-1), MFS (ZSM-57) and ZSM-48 family of microporous materials consisting of silicon, aluminium, oxygen and optionally boron. Advantageously in said first embodiment the catalyst (A1) is a crystalline silicate having a ratio Si/Al of at least about 100 or a dealuminated crystalline silicate.

The crystalline silicate having a ratio Si/Al of at least about 100 is advantageously selected among the MFI and the MEL.

The crystalline silicate having a ratio Si/Al of at least about 100 and the dealuminated crystalline silicate are essentially in H-form. It means that a minor part (less than about 50%) can carry metallic compensating ions e.g. Na, Mg, Ca, La, Ni, Ce, Zn, Co.

The dealuminated crystalline silicate is advantageously such as about 10% by weight of the aluminium is removed. Such dealumination is advantageously made by a steaming optionally followed by a leaching. The crystalline silicate having a ratio Si/Al of at least about 100 can be synthetized as such or it can be prepared by dealumination of a crystalline silicate at conditions effective to obtain a ratio Si/Al of at least about 100. Such dealumination is advantageously made by a steaming optionally followed by a leaching.

The three-letter designations “MFI” and “MEL” each representing a particular crystalline silicate structure type as established by the Structure Commission of the International Zeolite Association.

Examples of a crystalline silicate of the MFT type are the synthetic zeolite ZSM-5 and silicalite and other MFI type crystalline silicates known in the art. Examples of a crystalline silicate of the MEL family are the zeolite ZSM-11 and other MEL type crystalline silicates known in the art. Other examples are Boralite D and silicalite-2 as described by the International Zeolite Association (Atlas of zeolite structure types, 1987, Butterworths). The preferred crystalline silicates have pores or channels defined by ten oxygen rings and a high silicon/aluminium atomic ratio.

Crystalline silicates are microporous crystalline inorganic polymers based on a framework of XO₄ tetrahedra linked to each other by sharing of oxygen ions, where X may be trivalent (e.g. Al,B, . . . ) or tetravalent (e.g. Ge, Si, . . . ). The crystal structure of a crystalline silicate is defined by the specific order in which a network of tetrahedral units are linked together. The size of the crystalline silicate pore openings is determined by the number of tetrahedral units, or, alternatively, oxygen atoms, required to form the pores and the nature of the cations that are present in the pores. They possess a unique combination of the following properties: high internal surface area; uniform pores with one or more discrete sizes; ion exchangeability; good thermal stability; and ability to adsorb organic compounds. Since the pores of these crystalline silicates are similar in size to many organic molecules of practical interest, they control the ingress and egress of reactants and products, resulting in particular selectivity in catalytic reactions. Crystalline silicates with the MFI structure possess a bidirectional intersecting pore system with the following pore diameters: a straight channel along [010]:0.53-0.56 nm and a sinusoidal channel along [100]:0.51-0.55 nm. Crystalline silicates with the MEL structure possess a bidirectional intersecting straight pore system with straight channels along [100] having pore diameters of 0.53-0.54 nm.

In this specification, the term “silicon/aluminium atomic ratio” or “silicon/aluminium ratio” is intended to mean the framework Si/Al atomic ratio of the crystalline silicate. Amorphous Si and/or Al containing species, which could be in the pores are not a part of the framework. As explained hereunder in the course of a dealumination there is amorphous Al remaining in the pores, it has to be excluded from the overall Si/Al atomic ratio. The overall material referred above doesn't include the Si and Al species of the binder.

In a specific embodiment the catalyst preferably has a high silicon/aluminium atomic ratio, of at least about 100, preferably greater than about 150, more preferably greater than about 200, whereby the catalyst has relatively low acidity. The acidity of the catalyst can be determined by the amount of residual ammonia on the catalyst following contact of the catalyst with ammonia which adsorbs to the acid sites on the catalyst with subsequent ammonium desorption at elevated temperature measured by differential thermogravimetric analysis. Preferably, the silicon/aluminium ratio (Si/Al) ranges from about 100 to about 1000, most preferably from about 200 to about 1000. Such catalysts are known per se.

In a specific embodiment the crystalline silicate is steamed to remove aluminium from the crystalline silicate framework. The steam treatment is conducted at elevated temperature, preferably in the range of from 425 to 870° C., more preferably in the range of from 540 to 815° C. and at atmospheric pressure and at a water partial pressure of from 13 to 200 kPa. Preferably, the steam treatment is conducted in an atmosphere comprising from 5 to 100% steam. The steam atmosphere preferably contains from 5 to 100 vol % steam with from 0 to 95 vol % of an inert gas, preferably nitrogen. A more preferred atmosphere comprises 72 vol % steam and 28 vol % nitrogen i.e. 72 kPa steam at a pressure of one atmosphere. The steam treatment is preferably carried out for a period of from 1 to 200 hours, more preferably from 20 hours to 100 hours. As stated above, the steam treatment tends to reduce the amount of tetrahedral aluminium in the crystalline silicate framework, by forming alumina.

In a more specific embodiment the crystalline silicate catalyst is dealuminated by heating the catalyst in steam to remove aluminium from the crystalline silicate framework and extracting aluminium from the catalyst by contacting the catalyst with a complexing agent for aluminium to remove from pores of the framework alumina deposited therein during the steaming step thereby to increase the silicon/aluminium atomic ratio of the catalyst. The catalyst having a high silicon/aluminium atomic ratio for use in the catalytic process of the present invention is manufactured by removing aluminium from a commercially available crystalline silicate. By way of example a typical commercially available silicalite has a silicon/aluminium atomic ratio of around 120. In accordance with the present invention, the commercially available crystalline silicate is modified by a steaming process which reduces the tetrahedral aluminium in the crystalline silicate framework and converts the aluminium atoms into octahedral aluminium in the form of amorphous alumina. Although in the steaming step aluminium atoms are chemically removed from the crystalline silicate framework structure to form alumina particles, those particles cause partial obstruction of the pores or channels in the framework. This could inhibit the dehydration process of the present invention. Accordingly, following the steaming step, the crystalline silicate is subjected to an extraction step wherein amorphous alumina is removed from the pores and the micropore volume is, at least partially, recovered. The physical removal, by a leaching step, of the amorphous alumina from the pores by the formation of a water-soluble aluminium complex yields the overall effect of de-alumination of the crystalline silicate. In this way by removing aluminium from the crystalline silicate framework and then removing alumina formed there from the pores, the process aims at achieving a substantially homogeneous de-alumination throughout the whole pore surfaces of the catalyst. This reduces the acidity of the catalyst. The reduction of acidity ideally occurs substantially homogeneously throughout the pores defined in the crystalline silicate framework. Following the steam treatment, the extraction process is performed in order to de-aluminate the catalyst by leaching. The aluminium is preferably extracted from the crystalline silicate by a complexing agent which tends to form a soluble complex with alumina. The complexing agent is preferably in an aqueous solution thereof. The complexing agent may comprise an organic acid such as citric acid, formic acid, oxalic acid, tartaric acid, malonic acid, succinic acid, glutaric acid, adipic acid, maleic acid, phthalic acid, isophthalic acid, fumaric acid, nitrilotriacetic acid, hydroxyethylenediaminetriacetic acid, ethylenediaminetetracetic acid, trichloroacetic acid trifluoroacetic acid or a salt of such an acid (e.g. the sodium salt) or a mixture of two or more of such acids or salts. The complexing agent may comprise an inorganic acid such as nitric acid, halogenic acids, sulphuric acid, phosphoric acid or salts of such acids or a mixture of such acids. The complexing agent may also comprise a mixture of such organic and inorganic acids or their corresponding salts. The complexing agent for aluminium preferably forms a water-soluble complex with aluminium, and in particular removes alumina which is formed during the steam treatment step from the crystalline silicate. A particularly preferred complexing agent may comprise an amine, preferably ethylene diamine tetraacetic acid (EDTA) or a salt thereof, in particular the sodium salt thereof. In a preferred embodiment, the framework silicon/aluminium ratio is increased by this process to a value of from about 150 to 1000, more preferably at least 200. The leaching can also be made with a strong mineral acid such as HCl.

Following the aluminium leaching step, the crystalline silicate may be subsequently washed, for example with distilled water, and then dried, preferably at an elevated temperature, for example around 110° C.

Additionally, if during the preparation of the catalysts of the invention alkaline or alkaline earth metals have been used, the molecular sieve might be subjected to an ion-exchange step. Conventionally, ion-exchange is done in aqueous solutions using ammonium salts or inorganic acids.

Following the de-alumination step, the catalyst is thereafter calcined, for example at a temperature of from 400 to 800° C. at atmospheric pressure for a period of from 1 to 10 hours.

In another specific embodiment the crystalline silicate catalyst is mixed with a binder, preferably an inorganic binder, and shaped to a desired shape, e.g. pellets. The binder is selected so as to be resistant to the temperature and other conditions employed in the dehydration process of the invention. The binder is an inorganic material selected from clays, silica, metal silicate, metal oxides such as Zr0₂ and/or metals, or gels including mixtures of silica and metal oxides. The binder is preferably alumina-free. If the binder which is used in conjunction with the crystalline silicate is itself catalytically active, this may alter the conversion and/or the selectivity of the catalyst. Inactive materials for the binder may suitably serve as diluents to control the amount of conversion so that products can be obtained economically and orderly without employing other means for controlling the reaction rate. It is desirable to provide a catalyst having a good crush strength. This is because in commercial use, it is desirable to prevent the catalyst from breaking down into powder-like materials. Such clay or oxide binders have been employed normally only for the purpose of improving the crush strength of the catalyst. A particularly preferred binder for the catalyst of the present invention comprises silica. The relative proportions of the finely divided crystalline silicate material and the inorganic oxide matrix of the binder can vary widely. Typically, the binder content ranges from 5 to 95% by weight, more typically from 20 to 50% by weight, based on the weight of the composite catalyst. Such a mixture of crystalline silicate and an inorganic oxide binder is referred to as a formulated crystalline silicate. In mixing the catalyst with a binder, the catalyst may be formulated into pellets, extruded into other shapes, or formed into spheres or a spray-dried powder. Typically, the binder and the crystalline silicate catalyst are mixed together by a mixing process. In such a process, the binder, for example silica, in the form of a gel is mixed with the crystalline silicate catalyst material and the resultant mixture is extruded into the desired shape, for example cylindic or multi-lobe bars. Spherical shapes can be made in rotating granulators or by oil-drop technique. Small spheres can further be made by spray-drying a catalyst-binder suspension. Thereafter, the formulated crystalline silicate is calcined in air or an inert gas, typically at a temperature of from 200 to 900° C. for a period of from 1 to 48 hours. The binder preferably does not contain any aluminium compounds, such as alumina. This is because as mentioned above the preferred catalyst for use in the invention is de-aluminated to increase the silicon/aluminium ratio of the crystalline silicate. The presence of alumina in the binder yields other excess alumina if the binding step is performed prior to the aluminium extraction step. If the aluminium-containing binder is mixed with the crystalline silicate catalyst following aluminium extraction, this re-aluminates the catalyst.

In addition, the mixing of the catalyst with the binder may be carried out either before or after the steaming and extraction steps.

According to a second advantageous embodiment the catalyst (A1) is a crystalline silicate catalyst having a monoclinic structure, which has been produced by a process comprising providing a crystalline silicate of the MFI-type having a silicon/aluminium atomic ratio lower than 80; treating the crystalline silicate with steam and thereafter leaching aluminium from the zeolite by contact with an aqueous solution of a leachant to provide a silicon/aluminium atomic ratio in the catalyst of at least 180 whereby the catalyst has a monoclinic structure.

Preferably, in the steam treatment step the temperature is from 425 to 870° C., more preferably from 540 to 815° C., and at a water partial pressure of from 13 to 200 kPa.

Preferably, the aluminium is removed by leaching to form an aqueous soluble compound by contacting the zeolite with an aqueous solution of a complexing agent for aluminium which tends to form a soluble complex with alumina.

In accordance with this preferred process for producing monoclinic crystalline silicate, the starting crystalline silicate catalyst of the MFI-type has an orthorhombic symmetry and a relatively low silicon/aluminium atomic ratio which can have been synthesized without any organic template molecule and the final crystalline silicate catalyst has a relatively high silicon/aluminium atomic ratio and monoclinic symmetry as a result of the successive steam treatment and aluminium removal. After the aluminium removal step, the crystalline silicate may be ion exchanged with ammonium ions. It is known in the art that such MFI-type crystalline silicates exhibiting orthorhombic symmetry are in the space group Pnma. The x-ray diffraction diagram of such an orthorhombic structure has one peak at d=around 0.365 nm, d=around 0.305 nm and d=around 0.300 nm (see EP-A-0146524).

The starting crystalline silicate has a silicon/aluminium atomic ratio lower than 80. A typical ZSM-5 catalyst has 3.08 wt % Al₂O₃, 0.062 wt % Na₂0, and is 100% orthorhombic. Such a catalyst has a silicon/aluminium atomic ratio of 26.9.

The steam treatment step is carried out as explained above. The steam treatment tends to reduce the amount of tetrahedral aluminium in the crystalline silicate framework by forming alumina. The aluminium leaching or extraction step is carried out as explained above. In the aluminium leaching step, the crystalline silicate is immersed in the acidic solution or a solution containing the complexing agent and is then preferably heated, for example heated at reflux conditions (at boiling temperature with total return of condensed vapours), for an extended period of time, for example 18 hours. Following the aluminium leaching step, the crystalline silicate is subsequently washed, for example with distilled water, and then dried, preferably at an elevated temperature, for example around 110° C. Optionally, the crystalline silicate is subjected to ion exchange with ammonium ions, for example by immersing the crystalline silicate in an aqueous solution of NH₄Cl.

Finally, the catalyst is calcined at an elevated temperature, for example at a temperature of at least 400° C. The calcination period is typically around 3 hours.

The resultant crystalline silicate has monoclinic symmetry, being in the space group P2₁/n. The x-ray diffraction diagram of the monoclinic structure exhibits three doublets at d=around 0.36, 0.31 and 0.19 nm. The presence of such doublets is unique for monoclinic symmetry. More particularly, the doublet at d=around 0.36, comprises two peaks, one at d=0.362 nm and one at d=0.365 nm. In contrast, the orthorhombic structure has a single peak at d=0.365 nm.

The presence of a monoclinic structure can be quantified by comparing the x-ray diffraction line intensity at d=around 0.36 nm. When mixtures of MFI crystalline silicates with pure orthorhombic and pure monoclinic structure are prepared, the composition of the mixtures can be expressed as a monoclinicity index (in %). The x-ray diffraction patterns are recorded and the peak height at d=0.362 nm for monoclinicity and d=0.365 nm for orthorhombicity is measured and are denoted as Im and Io respectively. A linear regression line between the monoclinicity index and the Im/Io gives the relation needed to measure the monoclinicity of unknown samples. Thus the monoclinicity index %=(axIm/Io-b)×100, where a and b are regression parameters.

The such monoclinic crystalline silicate can be produced having a relatively high silicon/aluminium atomic ratio of at least 100, preferably greater than about 200 preferentially without using an organic template molecule during the crystallisation step. Furthermore, the crystallite size of the monoclinic crystalline silicate can be kept relatively low, typically less than 1 micron, more typically around 0.5 microns, since the starting crystalline silicate has low crystallite size which is not increased by the subsequent process steps. Accordingly, since the crystallite size can be kept relatively small, this can yield a corresponding increase in the activity of the catalyst. This is an advantage over known monoclinic crystalline silicate catalysts where typically the crystallite size is greater than 1 micron as they are produced in presence of an organic template molecule and directly having a high Si/Al ratio which inherently results in larger crystallites sizes.

According to a third advantageous embodiment the catalyst (Al) is a P-modified zeolite (Phosphorus-modified zeolite). Said phosphorus modified molecular sieves can be prepared based on MFI, MOR, MEL, clinoptilolite or FER crystalline aluminosilicate molecular sieves having an initial Si/Al ratio advantageously between 4 and 500. The P-modified zeolites of this recipe can be obtained based on cheap crystalline silicates with low Si/Al ratio (below 30).

By way of example said P-modified zeolite is made by a process comprising in that order:
selecting a zeolite (advantageously with Si/Al ratio between 4 and 500) among H⁺ or NH₄+-form of MFI, MEL, FER, MOR, clinoptilolite;
introducing P at conditions effective to introduce advantageously at least 0.05 wt % of P;
separation of the solid from the liquid if any;
an optional washing step or an optional drying step or an optional drying step followed by a washing step;
a calcination step; the catalyst of the XTO and the catalyst of the OCP being the same or different.

The zeolite with low Si/Al ratio has been made previously with or without direct addition of an organic template.

Optionally the process to make said P-modified zeolite comprises the steps of steaming and leaching. The method consists in steaming followed by leaching. It is generally known by the persons in the art that steam treatment of zeolites, results in aluminium that leaves the zeolite framework and resides as aluminiumoxides in and outside the pores of the zeolite. This transformation is known as dealumination of zeolites and this term will be used throughout the text. The treatment of the steamed zeolite with an acid solution results in dissolution of the extra-framework aluminiumoxides. This transformation is known as leaching and this term will be used throughout the text. Then the zeolite is separated, advantageously by filtration, and optionally washed. A drying step can be envisaged between filtering and washing steps. The solution after the washing can be either separated, by way of example, by filtering from the solid or evaporated.

P can be introduced by any means or, by way of example, according to the recipe described in U.S. Pat. No. 3,911,041, U.S. Pat. No. 5,573,990 and U.S. Pat. No. 6,797,851.

The catalyst (A1) made of a P-modified zeolite can be the P-modified zeolite itself or it can be the P-modified zeolite formulated into a catalyst by combining with other materials that provide additional hardness or catalytic activity to the finished catalyst product.

The separation of the liquid from the solid is advantageously made by filtering at a temperature between 0-90° C., centrifugation at a temperature between 0-90° C., evaporation or equivalent.

Optionally, the zeolite can be dried after separation before washing. Advantageously said drying is made at a temperature between 40-600° C., advantageously for 1-10 h. This drying can be processed either in a static condition or in a gas flow. Air, nitrogen or any inert gases can be used.

The washing step can be performed either during the filtering (separation step) with a portion of cold (<40° C.) or hot water (>40 but <90° C.) or the solid can be subjected to a water solution (1 kg of solid/4 liters water solution) and treated under reflux conditions for 0.5-10 h followed by evaporation or filtering.

Final calcination step is performed advantageously at the temperature 400-700° C. either in a static condition or in a gas flow. Air, nitrogen or any inert gases can be used.

According to a specific embodiment of this third advantageous embodiment of the invention the phosphorous modified zeolite is made by a process comprising in that order:
selecting a zeolite (advantageously with Si/Al ratio between 4 and 500, from 4 to 30 in a specific embodiment) among H⁺ or NH₄⁺-form of MFI, MEL, FER, MOR, clinoptilolite;
steaming at a temperature ranging from 400 to 870° C. for 0.01-200 h;
leaching with an aqueous acid solution at conditions effective to remove a substantial part of Al from the zeolite;
introducing P with an aqueous solution containing the source of P at conditions effective to introduce advantageously at least 0.05 wt % of P;
separation of the solid from the liquid;
an optional washing step or an optional drying step or an optional drying step followed by a washing step;
a calcination step.

Optionally between the steaming step and the leaching step there is an intermediate step such as, by way of example, contact with silica powder and drying.

Advantageously the selected MFI, MEL, FER, MOR, elinoptilolite (or H⁺ or NH₄⁺-form MFI, MEL, FER, MOR, clinoptilolite) has an initial atomic ratio Si/Al of 100 or lower and from 4 to 30 in a specific embodiment. The conversion to the H⁺ or NH₄⁺-form is known per se and is described in U.S. Pat. No. 3,911,041 and U.S. Pat. No. 5,573,990.

Advantageously the final P-content is at least 0.05 wt % and preferably between 0.3 and 7 w %. Advantageously at least 10% of Al, in respect to parent zeolite MFI, MEL, FER, MOR and clinoptilolite, have been extracted and removed from the zeolite by the leaching.

Then the zeolite either is separated from the washing solution or is dried without separation from the washing solution. Said separation is advantageously made by filtration. Then the zeolite is calcined, by way of example, at 400° C. for 2-10 hours.

In the steam treatment step, the temperature is preferably from 420 to 870° C., more preferably from 480 to 760° C. The pressure is preferably atmospheric pressure and the water partial pressure may range from 13 to 100 kPa. The steam atmosphere preferably contains from 5 to 100 vol % steam with from 0 to 95 vol % of an inert gas, preferably nitrogen. The steam treatment is preferably carried out for a period of from 0.01 to 200 hours, advantageously from 0.05 to 200 hours, more preferably from 0.05 to 50 hours. The steam treatment tends to reduce the amount of tetrahedral aluminium in the crystalline silicate framework by forming alumina.

The leaching can be made with a strong acid such as HCl or an organic acid such as citric acid, formic acid, oxalic acid, tartaric acid, malonic acid, succinic acid, glutaric acid, adipic acid, maleic acid, phthalic acid, isophthalic acid, fumaric acid, nitrilotriacetic acid, hydroxyethylenediaminetriacetic acid, ethylenediaminetetracetic acid, trichloroacetic acid trifluoroacetic acid or a salt of such an acid (e.g. the sodium salt) or a mixture of two or more of such acids or salts. The other inorganic acids may comprise an inorganic acid such as nitric acid, hydrochloric acid, methansulfuric acid, phosphoric acid, phosphonic acid, sulfuric acid or a salt of such an acid (e.g. the sodium or ammonium salts) or a mixture of two or more of such acids or salts.

The residual P-content is adjusted by P-concentration in the aqueous acid solution containing the source of P, drying conditions and a washing procedure if any. A drying step can be envisaged between filtering and washing steps.

Said P-modified zeolite can be used as itself as a catalyst. In another embodiment it can be formulated into a catalyst by combining with other materials that provide additional hardness or catalytic activity to the finished catalyst product. Materials which can be blended with the P-modified zeolite can be various inert or catalytically active materials, or various binder materials. These materials include compositions such as kaolin and other clays, various forms of rare earth metals, phosphates, alumina or alumina sol, titania, zirconia, quartz, silica or silica sol, and mixtures thereof. These components are effective in densifying the catalyst and increasing the strength of the formulated catalyst. The catalyst may be formulated into pellets, spheres, extruded into other shapes, or formed into a spray-dried particles. The amount of P-modified zeolite which is contained in the final catalyst product ranges from 10 to 90 weight percent of the total catalyst, preferably 20 to 70 weight percent of the total catalyst.

The reactor (II) is employed under particular reaction conditions whereby the catalytic cracking of the olefins readily proceeds. Different reaction pathways can occur on the catalyst. Olefinic catalytic cracking may be understood to comprise a process yielding shorter molecules via bond breakage.

In the catalytic cracking process of the OCP reactor, the process conditions are selected in order to provide high selectivity towards propylene or ethylene, as desired, a stable olefin conversion over time, and a stable olefinic product distribution in the effluent. Such objectives are favoured with a low pressure, a high inlet temperature and a short contact time, all of which process parameters are interrelated and provide an overall cumulative effect.

The process conditions are selected to disfavour hydrogen transfer reactions leading to the formation of paraffins, aromatics and coke precursors. The process operating conditions thus employ a high space velocity, a low pressure and a high reaction temperature. The LHSV ranges from 0.5 to 30 hr⁻¹, preferably from 1 to 30 hr⁻¹. The olefin partial pressure ranges from 0.1 to 2 bars, preferably from 0.5 to 1.5 bars (absolute pressures referred to herein). A particularly preferred olefin partial pressure is atmospheric pressure (i.e. 1 bar). The feedstock of the reactor (II) is preferably fed at a total inlet pressure sufficient to convey the feedstocks through the reactor (II). Said feedstock may be fed undiluted or diluted in an inert gas, e.g. nitrogen or steam. Preferably, the total absolute pressure in the reactor ranges from 0.5 to 10 bars. The use of a low olefin partial pressure, for example atmospheric pressure, tends to lower the incidence of hydrogen transfer reactions in the cracking process, which in turn reduces the potential for coke formation which tends to reduce catalyst stability. The cracking of the olefins is preferably performed at an inlet temperature of the feedstock of from 400° to 650° C., more preferably from 450° to 600° C., yet more preferably from 540° C. to 590° C. In order to maximize the amount of ethylene and propylene and to minimize the production of methane, aromatics and coke, it is desired to minimize the presence of diolefins in the feed, Diolefin conversion to monoolefin hydrocarbons may be accomplished with a conventional selective hydrogenation process such as disclosed in U.S. Pat. No. 4,695,560 hereby incorporated by reference.

The OCP reactor can be a fixed bed reactor, a moving bed reactor or a fluidized bed reactor. A typical fluid bed reactor is one of the FCC type used for fluidized-bed catalytic cracking in the oil refinery. A typical moving bed reactor is of the continuous catalytic reforming type. As described above, the process may be performed continuously using a pair of parallel “swing” reactors. The cracking process in reactor (II) is endothermic; therefore, the reactor should be adapted to supply heat as necessary to maintain a suitable reaction temperature. Several reactors may be used in series with interheating between the reactors in order to supply the required heat to the reaction. Each reactor does a part of the conversion of the feedstock. Online or periodic regeneration of the catalyst may be provided by any suitable means known in the art.

The various preferred catalysts of the OCP reactor have been found to exhibit high stability, in particular being capable of giving a stable propylene yield over several days, e.g. up to ten days. This enables the olefin cracking process to be performed continuously in two parallel “swing” reactors wherein when one reactor is in operation, the other reactor is undergoing catalyst regeneration, The catalyst can be regenerated several times.

### DESCRIPTION OF THE DRAWINGS

FIG. 1 shows a flow diagram of a naphtha cracker with a front-end de-methaniser configuration. The naphtha feedstock is sent (1) to the furnaces (2) where it is cracked into lighter components. The furnace effluent is sent to the section (3) comprising the primary fractionator and the quench section to cool down the effluent before entering into the compression section (4), including the acid gas removal unit (AGR) and gas driers. From each of the former sections the condensables are collected in the gasoline stripper (5) in which the light ends flow back to the compression section (4). The dried effluent is sent to the de-methaniser (7) where a mixture of hydrogen and methane (8) is separated from C₂+ hydrocarbons (10). These C₂+ hydrocarbons are further separated in the de-ethaniser (11) into an overhead stream containing the C₂ hydrocarbons (12) and a bottom stream containing the C₃+ hydrocarbons (13). The C₂ hydrocarbons can further be purified by selective hydrogenation of the acetylene and subsequently separated in a C₂ splitter into polymer grade ethylene and ethane rich stream. The C₃+ hydrocarbons (13 & 6a) are next separated in the de-propaniser (14) in an overhead stream of the C₃ hydrocarbons (15) and a bottom stream containing the C₄+ hydrocarbons (16). optionally (15) is sent to a MAPD (methyl acetylene propadiene) removing unit (not shown in FIG. 1) and then to a C3 splitter to produce a propylene polymer grade. Depending on the performance of the gasoline stripper (5), the stripper bottom product can be sent to the de-propaniser (14), de-butaniser (20) or de-pentaniser (23). The C₄+ hydrocarbons (16 & 6b) are sent to the de-butaniser (20), producing raw C4's (21) and raw pyrolysis gasoline (22). The raw pyrolysis gasoline (22 & 6c) is first stabilised in a “first stage hydrogenation” reactor (23) and next (24) sent to a de-pentaniser (25) where it is split into C₅ non-aromatic hydrocarbons (26) and aromatic rich C₆+ hydrocarbons (27). These C₆+ hydrocarbons can be further treated in order to recover benzene, toluene and xylenes.

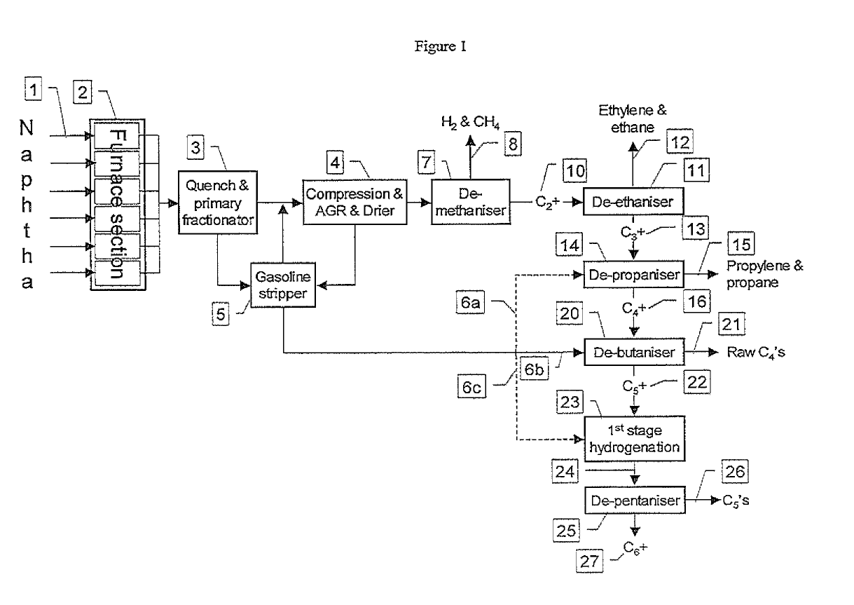

FIG. 2 shows a flow diagram of the naphtha cracker with a front-end de-methaniser configuration that is able to run under low-severity conditions by a synergetic integration with an olefin cracking process and hence maximising the production of propylene. The feedstock to the olefin cracking consist of the raw C₄'s (30), the C₅ non-aromatic hydrocarbons (26), part of the gasoline stripper bottom product (32) and optionally imported C₄+ olefinic hydrocarbons (33). These feedstock's are first treated in a selective hydrogenation reactor (34) to convert substantially the dienes and acetylenes into their corresponding olefins. When the naphtha cracker operates under low-severity conditions, more C₃+ hydrocarbons (13) as bottom of the de-ethaniser (11) and more stripped gasoline (6) are produced resulting in a bottleneck for the de-propaniser (14) and subsequent separation units. The stripped gasoline (6), having substantially little or no propylene can be sent to the selective hydrogenation reactor (34), whereas the excess C₃+ hydrocarbons (46), having high amounts of propylene has to be sent to the backend section of the olefin cracking process. Once the feedstock is selectively hydrogenated it is sent to the olefin cracking reactor (40). The effluent (41) flows to the rerun column (42) where the C₆+ hydrocarbons (43) are purged as bottom stream and the overhead (44) is sent together with the excess C₃+ hydrocarbons (46) coming from the steamcracker de-ethaniser (11) to the de-propaniser (45). The de-propaniser produces a bottom stream (47) C₄+ hydrocarbons that are for a part recycled (35) back to the selective hydrogenation reactor (34), the remaining C₄+ hydrocarbons (51) are sent to the de-butaniser (60) where they are split into C₅'s hydrocarbons (61) and C₄'s hydrocarbons (62). The de-propaniser overhead (48) is sent to the de-ethaniser (70) where they are split into C₂− hydrocarbons (72) and C₃'s hydrocarbons (71). The C₂− hydrocarbons (72) are further split in the de-methaniser (80) where they are split in hydrogen&methane (82) and C₂'s hydrocarbons (81). The C₂'s hydrocarbons (81) can be further purified by selective hydrogenation of the contained acetylene and by a C₂ splitter. The C₃'s hydrocarbons (71) can be further purified by selective hydrogenation of the contained methylacetylene and propadiene and by a C₃ splitter. The hydrogen&methane (82) can be further valorised by methanation to remove carbon monoxide and separation of the hydrogen from methane.

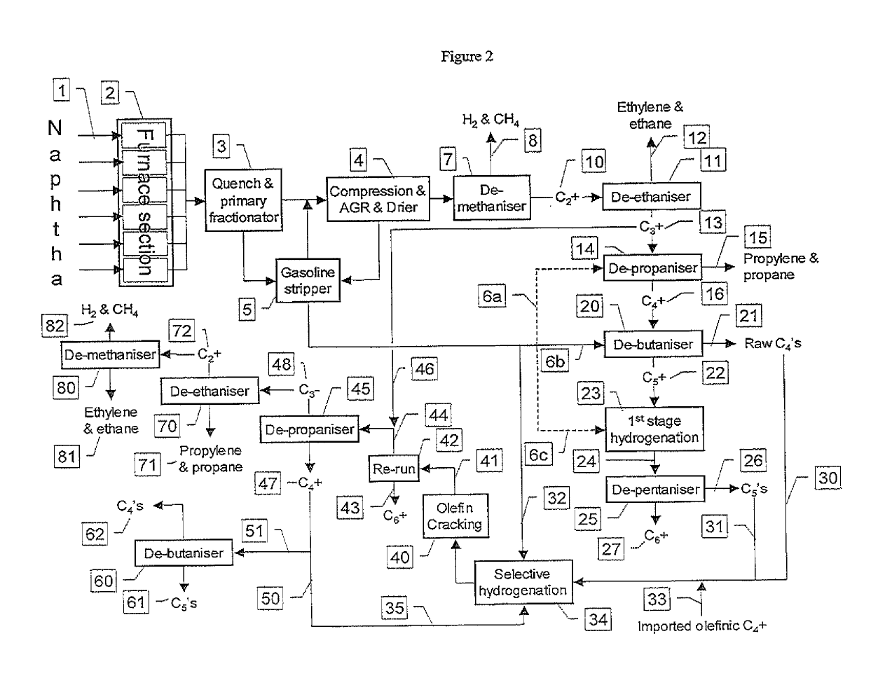

FIG. 3 shows a flow diagram of a naphtha cracker with a front-end de-methaniser configuration similar to the one described for FIG. 1. The difference is that the C₃'s hydrocarbons (15) are sent to a MAPD splitter (17) that produces a bottom C₃'s hydrocarbons stream (19) enriched in methylacetylene, propadiene and some C₄ hydrocarbons (commonly called tetrene fraction). The overhead consists of C₃'s hydrocarbons (18) that can be further purified by selective hydrogenation of the contained methylacetylene and propadiene and by a C₃ splitter.

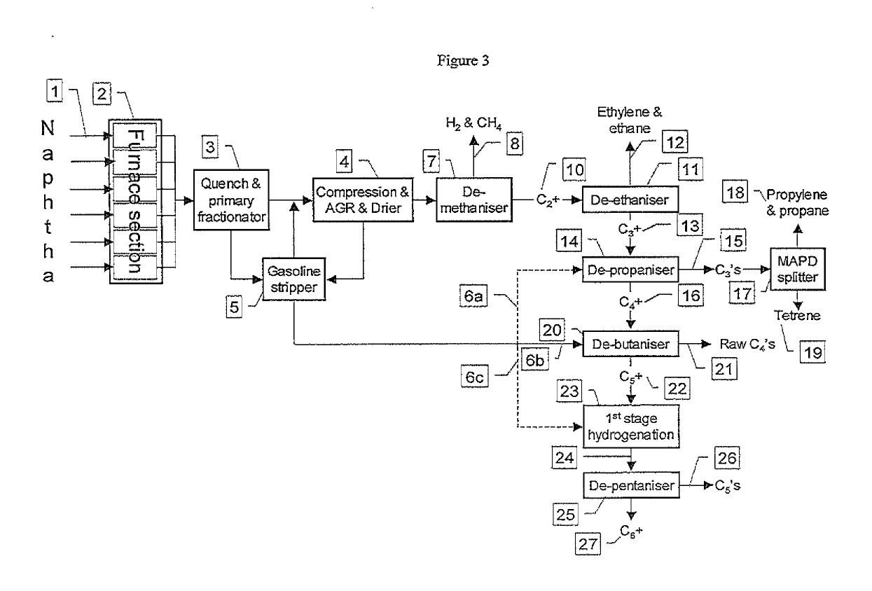

FIG. 4 shows a flow diagram of a naphtha cracker with a front-end de-methaniser configuration that is able to run under low-severity conditions by a synergetic integration with an olefin cracking process and hence maximising the production of propylene, similar to the one described for FIG. 2. The difference is that the C₃'s hydrocarbons (15) are sent to a MAPD splitter that produces a bottom C₃'s hydrocarbons stream (19) enriched in methylacetylene, propadiene and some C₄ hydrocarbons (commonly called tetrene fraction). This tetrene fraction (90) and excess C₃+ hydrocarbons (46), both having high amounts of propylene have to be sent to the backend section of the olefin cracking process, namely to the de-propaniser (45). The MAPD overhead consists of C₃'s hydrocarbons (18) that can be further purified by selective hydrogenation of the contained methylacetylene and propadiene and by a C₃ splitter.

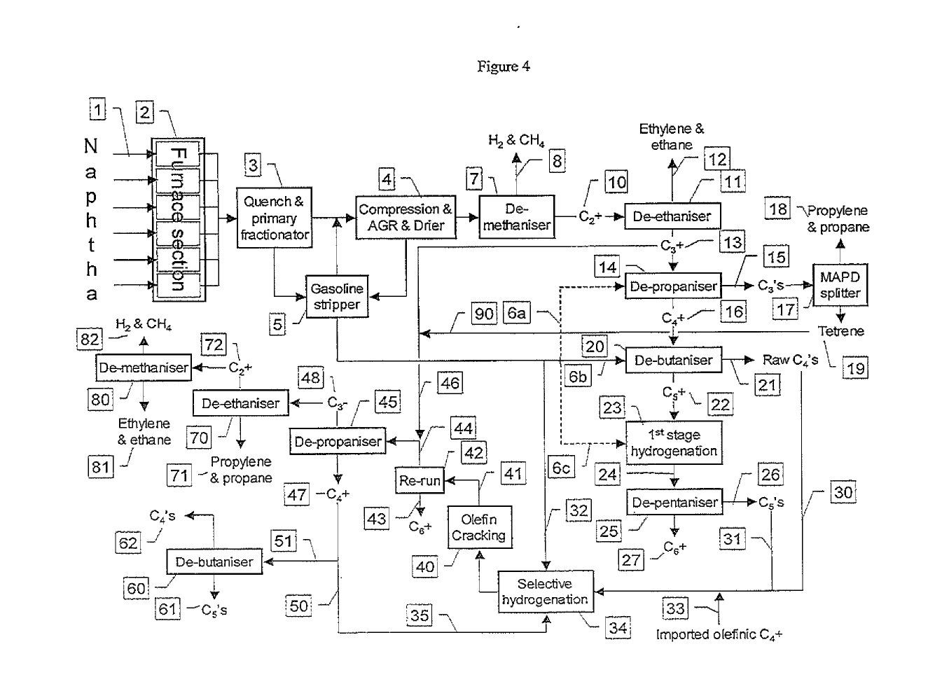

FIG. 5 shows a flow diagram of a naphtha cracker with a front-end de-ethaniser configuration. The naphtha feedstock is sent (1) to the furnaces (2) where it is cracked into lighter components. The furnace effluent is sent to the section (3) comprising the primary fractionator and the quench section to cool down the effluent before entering into the compression section (4), including the acid gas removal unit (AGR) and gas driers. From each of the former sections the condensables are collected in the gasoline stripper (5) in which the light ends flow back to the compression section (4). The dried effluent is sent to the de-ethaniser (11) and separated into an overhead stream containing the C₂− hydrocarbons (10) and a bottom stream containing the C₃+ hydrocarbons (13). The C₂− hydrocarbons (10) flow to a de-methaniser (7) where a mixture of hydrogen and methane (8) is separated from C₂'s hydrocarbons (12). The C₂ hydrocarbons (12) can further be purified by selective hydrogenation of the acetylene and subsequently separated in a C₂ splitter into polymer grade ethylene and ethane rich stream. The C₃+ hydrocarbons (13 & 6a) are next separated in the de-propaniser (14) in an overhead stream of the C₃ hydrocarbons (15) and a bottom stream containing the C₄+ hydrocarbons (16). Depending on the performance of the gasoline stripper (5), the stripper bottom product can be sent to the de-propaniser (14), de-butaniser (20) or de-pentaniser (23). The C₄+ hydrocarbons (16 & 6b) are sent to the de-butaniser (20), producing raw C4's (21) and raw pyrolysis gasoline (22). The raw pyrolysis gasoline (22 & 6c) is first stabilised in a “first stage hydrogenation” reactor (23) and next (24) sent to a de-pentaniser (25) where it is split into C₅ non-aromatic hydrocarbons (26) and aromatic rich C₆+ hydrocarbons (27). These C₆+ hydrocarbons can be further treated in order to recover benzene, toluene and xylenes.

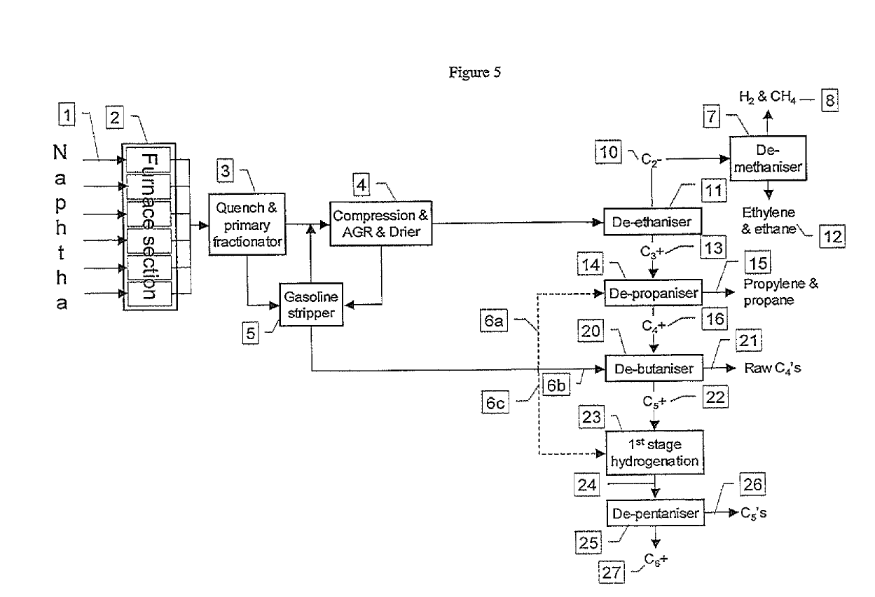

FIG. 6 shows a flow diagram of the naphtha cracker with a front-end de-ethaniser configuration that is able to run under low-severity conditions by a synergetic integration with an olefin cracking process and hence maximising the production of propylene. The feedstock to the olefin cracking consist of the raw C4's (30), the C₅ non-aromatic hydrocarbons (26), part of the gasoline stripper bottom product (32) and optionally imported C₄+ olefinic hydrocarbons (33). These feedstock's are first treated in a selective hydrogenation reactor (34) to convert substantially the dienes and acetylenes into their corresponding olefins. When the naphtha cracker operates under low-severity conditions, more C₃+ hydrocarbons (13) as bottom of the de-ethaniser (11) and more stripped gasoline (6) are produced resulting in a bottleneck for the de-propaniser (14) and subsequent separation units. The stripped gasoline (6), having substantially little or no propylene can be sent to the selective hydrogenation reactor (34), whereas the excess C₃+ hydrocarbons (46), having high amounts of propylene has to be sent to the backend section of the olefin cracking process. Once the feedstock is selectively hydrogenated it is sent to the olefin cracking reactor (40). The effluent (41) flows to the rerun column (42) where the C₆+ hydrocarbons (43) are purged as bottom stream and the overhead (44) is sent together with the excess C₃+ hydrocarbons (46) coming from the steamcracker de-ethaniser (11) to the de-propaniser (45). The de-propaniser produces a bottom stream (47) C₄+ hydrocarbons that are for a part recycled (35) back to the selective hydrogenation reactor (34), the remaining C₄+ hydrocarbons (51) are sent to the de-butaniser (60) where they are split into C₅'s hydrocarbons (61) and C₄'s hydrocarbons (62). The de-propaniser overhead (48) is sent to the de-ethaniser (70) where they are split into C₂− hydrocarbons (72) and C₃'s hydrocarbons (71). The C₂− hydrocarbons (72) are further split in the de-methaniser (80) where they are split in hydrogen&methane (82) and C₂'s hydrocarbons (81). The C₂'s hydrocarbons (81) can be further purified by selective hydrogenation of the contained acetylene and by a C2 splitter. The C₃'s hydrocarbons (71) can be further purified by selective hydrogenation of the contained methylacetylene and propadiene and by a C₃ splitter. The hydrogen&methane (82) can be further valorised by methanation to remove carbonmonoxide and separation of the hydrogen from methane.

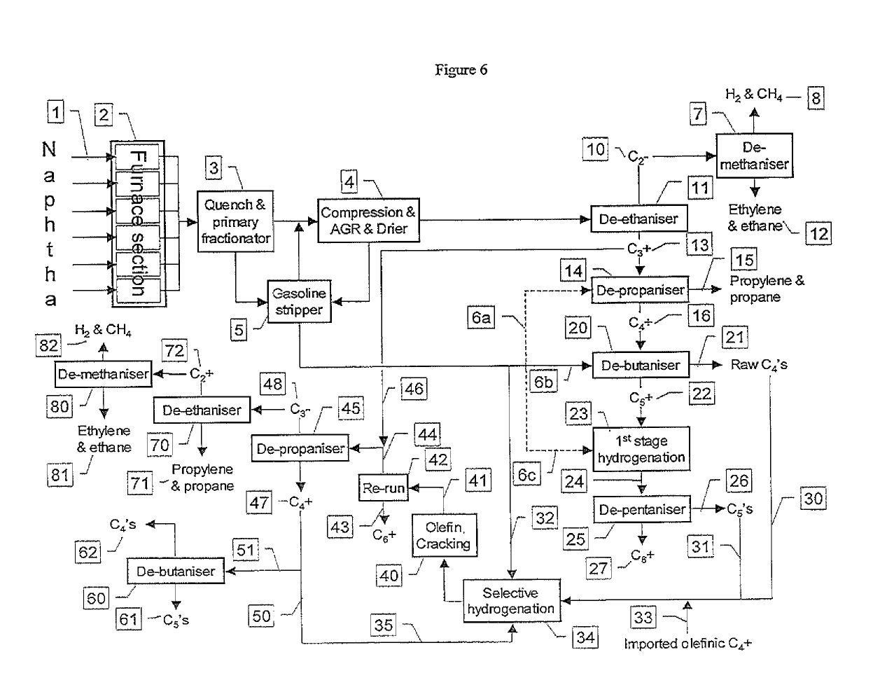

The table below shows the impact of operating a steamcracker under low severity conditions. The entries 1 to 4 show the effect of operating at lower severity from 0.3 to 0.6 (propylene to ethylene ratio) without changing the naphtha throughput and steam to naphtha ratio. The ethylene production rate decreases while the propylene production rate increases and at the same time the furnace duty is reduced and also the coking rate. Entry 5 is an improved case where the naphtha throughput could be increased up to a level where the same pressure drop is reached as for the case of entry 2, which can be considered as a reference case. For entry 6 and 7, the steam to naphtha ratio has been reduced while maintaining the furnace duty, pressure drop and coking rate not higher than for the case in entry 2. Entry 7 shows that for the same ethylene production rate and a similar fuel gas make, 130% of the reference (entry 2) propylene production rate could be accomplished. It shows also that 138 and 197% of the reference production rate of respectively raw C₄'s and C₅'s are produced. The heavier cracking components are the same or are reduced compared to entry 2. This table demonstrates that the technical constraints, created by the low severity operation, can be solved by added a new olefin cracking process that will crack the C₄'s and C₅'s, while the on-purpose propylene purification section of the olefin cracking process can also handle the incremental propylene produced on the steamcracker running under low severity conditions.

| | | 1 | 2 | 3 | 4 | 5 | 6 | 7 | 8 |
|---|---|---|---|---|---|---|---|---|---|
| THROUGHPUT | TON/HR | 15.5 | 15.5 | 15.5 | 15.5 | 15.8 | 16.6 | 17.1 | 15.5 |
| STEAM DILUTION | KG/KG | 0.40 | 0.40 | 0.40 | 0.40 | 0.40 | 0.35 | 0.30 | 0.40 |
| INLET RADIATION TEMP | ° C. | 530.0 | 530.0 | 530.0 | 530.0 | 530.0 | 530.0 | 530.0 | 530.0 |
| COIL OUTLET TEMP | ° C. | 897.9 | 866.9 | 847.3 | 837.6 | 838.3 | 839.0 | 838.9 | 828.0 |
| COIL INLET PRESSURE | BARA | 3.0 | 3.0 | 3.0 | 3.0 | 3.0 | 3.0 | 3.0 | 3.0 |
| COIL OUTLET PRESSURE | BARA | 2.1 | 2.1 | 2.1 | 2.1 | 2.1 | 2.1 | 2.1 | 2.1 |
| PRESSURE DROP COIL | BARA | 0.87 | 0.84 | 0.82 | 0.82 | 0.84 | 0.84 | 0.80 | 0.81 |
| THERMAL DUTY | MWATT | 14.9 | 14.0 | 13.4 | 13.1 | 13.4 | 13.8 | 14.0 | 12.8 |
| Coking rate at location of Max | [mm/month] | 8.1 | 5.9 | 4.7 | 4.1 | 4.2 | 4.6 | 4.9 | 3.6 |
| TMT | | | | | | | | | |
| P/E | WT %/WT % | 0.3 | 0.4 | 0.5 | 0.6 | 0.6 | 0.6 | 0.6 | 0.6 |
| P + E | WT % | 39.6 | 42.0 | 42.5 | 42.4 | 42.5 | 42.2 | 41.8 | 42.1 |
| YIELDS | | | | | | | | | |
| CO | tons/h | 0.024 | 0.016 | 0.012 | 0.010 | 0.010 | 0.010 | 0.009 | 0.008 |
| CO2 | tons/h | 0.003 | 0.001 | 0.001 | 0.001 | 0.001 | 0.001 | 0.000 | 0.001 |
| HYDROGEN | tons/h | 0.183 | 0.158 | 0.144 | 0.137 | 0.140 | 0.146 | 0.149 | 0.130 |
| METHANE | tons/h | 3.153 | 2.780 | 2.536 | 2.412 | 2.455 | 2.612 | 2.729 | 2.284 |
| ACETYLENE | tons/h | 0.161 | 0.093 | 0.064 | 0.053 | 0.054 | 0.055 | 0.054 | 0.043 |
| ETHYLENE | tons/h | 4.721 | 4.487 | 4.252 | 4.111 | 4.194 | 4.373 | 4.463 | 3.954 |
| ETHANE | tons/h | 0.456 | 0.576 | 0.625 | 0.641 | 0.652 | 0.710 | 0.761 | 0.653 |
| MAPD | tons/h | 0.148 | 0.133 | 0.112 | 0.100 | 0.103 | 0.106 | 0.106 | 0.089 |
| PROPYLENE | tons/h | 1.416 | 2.019 | 2.339 | 2.467 | 2.516 | 2.624 | 2.678 | 2.570 |
| PROPANE | tons/h | 0.036 | 0.058 | 0.070 | 0.076 | 0.077 | 0.083 | 0.089 | 0.080 |
| 1,3-BUTADIENE | tons/h | 0.615 | 0.712 | 0.749 | 0.758 | 0.775 | 0.799 | 0.802 | 0.759 |
| 1-BUTENE | tons/h | 0.072 | 0.137 | 0.196 | 0.230 | 0.235 | 0.241 | 0.240 | 0.265 |
| ISOBUTENE | tons/h | 0.153 | 0.302 | 0.407 | 0.458 | 0.467 | 0.485 | 0.493 | 0.506 |
| 2-BUTENE | tons/h | 0.038 | 0.080 | 0.118 | 0.139 | 0.142 | 0.146 | 0.147 | 0.162 |
| ISOBUTANE | tons/h | 0.001 | 0.005 | 0.009 | 0.011 | 0.011 | 0.011 | 0.012 | 0.013 |
| NBUTANE | tons/h | 0.007 | 0.028 | 0.051 | 0.065 | 0.066 | 0.068 | 0.067 | 0.080 |
| OTHER C4 | tons/h | 0.026 | 0.014 | 0.009 | 0.007 | 0.007 | 0.007 | 0.007 | 0.006 |
| C5 CUT | tons/h | 0.191 | 0.389 | 0.602 | 0.735 | 0.750 | 0.768 | 0.767 | 0.884 |
| C6 NONARO | tons/h | 0.033 | 0.098 | 0.199 | 0.278 | 0.283 | 0.285 | 0.279 | 0.379 |
| BENZENE | tons/h | 1.744 | 1.567 | 1.416 | 1.334 | 1.357 | 1.448 | 1.518 | 1.249 |
| C7 NONARO | tons/h | 0.004 | 0.017 | 0.042 | 0.065 | 0.066 | 0.066 | 0.065 | 0.096 |
| TOLUENE | tons/h | 0.503 | 0.514 | 0.494 | 0.478 | 0.486 | 0.521 | 0.550 | 0.458 |
| C8 NONARO | tons/h | 0.000 | 0.001 | 0.003 | 0.005 | 0.005 | 0.005 | 0.005 | 0.008 |
| ETHYLBENZENE/XYLENES | tons/h | 0.078 | 0.097 | 0.103 | 0.104 | 0.106 | 0.114 | 0.120 | 0.104 |
| STYRENE | tons/h | 0.331 | 0.218 | 0.162 | 0.138 | 0.140 | 0.151 | 0.160 | 0.116 |
| C9 NONARO | tons/h | 0.000 | 0.000 | 0.000 | 0.001 | 0.001 | 0.001 | 0.001 | 0.001 |
| C9 ARO | tons/h | 0.129 | 0.124 | 0.115 | 0.108 | 0.110 | 0.119 | 0.128 | 0.102 |
| C10 | tons/h | 0.296 | 0.259 | 0.221 | 0.200 | 0.204 | 0.221 | 0.237 | 0.179 |
| C11 | tons/h | 0.069 | 0.059 | 0.049 | 0.044 | 0.045 | 0.048 | 0.052 | 0.039 |
| C12+ | tons/h | 0.907 | 0.557 | 0.400 | 0.337 | 0.342 | 0.377 | 0.411 | 0.283 |
| THROUGHPUT | TON/HR | 15.5 | 15.5 | 15.5 | 16.5 | 15.8 | 16.6 | 17.1 | 15.5 |
| STEAM DILUTION | KG/KG | 0.40 | 0.40 | 0.40 | 0.40 | 0.40 | 0.35 | 0.30 | 0.40 |
| INLET RADIATION TEMP | ° C. | 530.0 | 530.0 | 530.0 | 530.0 | 530.0 | 530.0 | 530.0 | 530.0 |
| COIL OUTLET TEMP | ° C. | 897.9 | 866.9 | 847.3 | 837.6 | 838.3 | 839.0 | 838.9 | 828.0 |
| COIL INLET PRESSURE | BARA | 3.0 | 3.0 | 3.0 | 3.0 | 3.0 | 3.0 | 3.0 | 3.0 |
| COIL OUTLET PRESSURE | BARA | 2.1 | 2.1 | 2.1 | 2.1 | 2.1 | 2.1 | 2.1 | 2.1 |
| PRESSURE DROP COIL | BARA | 0.87 | 0.84 | 0.82 | 0.82 | 0.84 | 0.84 | 0.80 | 0.81 |
| THERMAL DUTY | MWATT | 14.9 | 14.0 | 13.4 | 13.1 | 13.4 | 13.8 | 14.0 | 12.8 |
| Coking rate at location of max | [mm/month] | 8.1 | 5.9 | 4.7 | 4.1 | 4.2 | 4.6 | 4.9 | 3.6 |
| TMT | | | | | | | | | |
| P/E | WT %/WT % | 0.3 | 0.4 | 0.5 | 0.6 | 0.6 | 0.6 | 0.6 | 0.6 |
| P + E | WT % | 39.6 | 42.0 | 42.5 | 42.4 | 42.5 | 42.2 | 41.8 | 42.1 |
| Fuel gas | tons/h | 3.363 | 2.955 | 2.693 | 2.559 | 2.606 | 2.768 | 2.887 | 2.423 |
| C2's | tons/h | 5.338 | 5.156 | 4.941 | 4.805 | 4.900 | 5.139 | 5.278 | 4.650 |
| C3's | tons/h | 1.601 | 2.210 | 2.521 | 2.643 | 2.896 | 2.813 | 2.872 | 2.739 |
| Raw C4's | tons/h | 0.914 | 1.278 | 1.539 | 1.667 | 1.703 | 1.758 | 1.769 | 1.790 |
| C5's | tons/h | 0.191 | 0.389 | 0.602 | 0.735 | 0.750 | 0.768 | 0.787 | 0.884 |
| C6-C11 | tons/h | 3.187 | 2.954 | 2.804 | 2.754 | 2.802 | 2.979 | 3.115 | 2.732 |
| C12+ | tons/h | 0.907 | 0.557 | 0.400 | 0.337 | 0.342 | 0.377 | 0.411 | 0.283 |
| % change in flow rate | | | | | | | | | |
| Fuel gas | | 113.8% | 100.0% | 91.1% | 86.6% | 88.2% | 93.7% | 97.7% | 82.0% |
| C2's | | 103.5% | 100.0% | 95.8% | 93.2% | 95.0% | 99.7% | 102.4% | 90.2% |
| C3's | | 72.4% | 100.0% | 114.1% | 119.6% | 122.0% | 127.3% | 130.0% | 124.0% |
| Raw C4's | | 71.5% | 100.0% | 120.4% | 130.4% | 133.3% | 137.5% | 138.4% | 140.0% |
| C5's | | 49.1% | 100.0% | 154.8% | 188.9% | 192.8% | 197.3% | 197.2% | 227.3% |
| C6-C11 | | 107.9% | 100.0% | 94.9% | 93.2% | 94.8% | 100.8% | 105.4% | 92.5% |
| C12+ | | 162.8% | 100.0% | 71.8% | 60.5% | 61.4% | 67.7% | 73.7% | 50.7% |

1. Method for debottlenecking an existing steam cracker unit of which the operation is modified from high severity to low severity operation, having a cracking zone and a fractionation zone, said fractionation zone comprising a gasoline stripper, a de-methaniser (I), a de-ethaniser (I) a de-propaniser (I) and a de-butaniser (I), said de-propaniser (I) receiving product from the bottom of the de-ethaniser (I) and optionally product from the bottom of the gasoline stripper (I),
wherein said debottlenecking method comprises the steps of:
a) adding a selective hydrogenation unit (II),
b) adding a cracking reactor (II) comprising a catalyst selective towards light olefins in the outlet,
c) adding a re-run column and a de-propaniser (II),
d) sending a part or all of the bottoms stream of the gasoline stripper (I) to the selective hydrogenation unit (II) and subsequently to the cracking reactor (II) at conditions effective to produce an outlet with an olefin content of lower molecular weight than that of the inlet,
e) sending a part of the bottoms stream of the de-ethaniser (I) to the de-propaniser (II), such as, not to overload the de-propaniser (I)
f) optionally sending a part or all of the overhead raw C₄ fraction of the de-butaniser (I) to the selective hydrogenation unit (II),
g) sending the cracking reactor (II) outlet to the re-run column to produce a C₆+ bottom stream and a C₁-C₅ overhead, sending said overhead to the de-propaniser to produce a C₁-C₃ overhead and a C₄+ bottom stream recycled in whole or in part to the selective hydrogenation unit (II), optionally withdrawing a part of said C₄+ bottom stream.

2. Method according to claim 1 for debottlenecking an existing steam cracker unit of which the operation is modified from high severity to low severity operation, having a cracking zone and a fractionation zone, said fractionation zone comprising a gasoline stripper, a de-methaniser (I), a de-ethaniser (I) a de-propaniser (I) and a de-butaniser (I), in which, said de-ethaniser (I) is producing,
an overhead stream comprising ethylene, ethane and optionally fuel gas,
a bottoms stream comprising C₃+ sent to the de-propaniser (I),
said de-propaniser (I) receiving product from the bottom of the de-ethaniser (I) and optionally product from the bottom of the gasoline stripper (I), said de-propaniser (I) producing,
an overhead stream sent to a MAPD removing unit (I) to produce propane and propylene,
a bottoms stream comprising C₄+ sent to the de-butaniser (I) to produce an overhead raw C₄ fraction and a bottom C₅+ fraction,
wherein said debottlenecking method comprises the steps of:
a) adding a selective hydrogenation unit (II),
b) adding a cracking reactor (II) comprising a catalyst selective towards light olefins in the outlet,
c) adding a re-run column and a de-propaniser (II),
d) sending a part or all of the bottoms stream of the gasoline stripper (I) to the selective hydrogenation unit (II) and subsequently to the cracking reactor (II) at conditions effective to produce an outlet with an olefin content of lower molecular weight than that of the inlet,
e) sending a part of the bottoms stream of the de-ethaniser (I) to the de-propaniser (II), such as, not to overload the de-propaniser (I)
f) optionally sending a part or all of the overhead raw C₄ fraction of the de-butaniser (I) to the selective hydrogenation unit (II),
g) sending the cracking reactor (II) outlet to the re-run column to produce a C₆+ bottom stream and a C₁-C₅ overhead, sending said overhead to the de-propaniser (II) to produce a C₁-C₃ overhead and a C₄+ bottom stream recycled in whole or in part to the selective hydrogenation unit (II), optionally withdrawing a part of said C₄+ bottom stream.

3. Method according to claim 2 for debottlenecking an existing steam cracker unit of which the operation is modified from high severity to low severity operation, having a cracking zone and a fractionation zone, said fractionation zone comprising a gasoline stripper, then a fractionation configuration with first a de-methaniser (I) (front-end de-methaniser), followed by a de-ethaniser (I) and followed by a de-propaniser (I) and a de-butaniser (I), in which, said de-ethaniser (I) is producing,
an overhead stream sent to a C₂ splitter (I) through a back-end acetylene converter (I) to separate ethylene and ethane,
a bottoms stream comprising C₃+ sent to the de-propaniser (I),
said de-propaniser (I) receiving product from the bottom of the de-ethaniser (I) and optionally product from the bottom of the gasoline stripper (I), said de-propaniser (I) producing,
an overhead stream sent to a MAPD removing unit (I) to produce propane and propylene,
a bottoms stream comprising C₄+ sent to the de-butaniser (I) to produce an overhead raw C₄ fraction and a bottom C₅+ fraction, optionally said C₅+ fraction is subsequently sent to a de-pentaniser (I) to produce an overhead C₅ fraction and a bottom C₆+ fraction,
wherein said debottlenecking method comprises the steps of:
a) adding a selective hydrogenation unit (II),
b) adding a cracking reactor (II) comprising a catalyst selective towards light olefins in the outlet,
c) adding a re-run column, a de-propaniser (II), a de-ethaniser (II), optionally a de-methaniser (II), optionally a MAPD conversion unit (II) and optionally a C₃ splitter (II) to separate propane and propylene,
d) sending a part or all of the bottoms stream of the gasoline stripper (I) to the selective hydrogenation unit (II) and subsequently to the cracking reactor (II) at conditions effective to produce an outlet with an olefin content of lower molecular weight than that of the inlet,
e) sending a part of the bottoms stream of the de-ethaniser (I) to the de-propaniser (II), such as, not to overload the de-propaniser (I)
f) optionally sending a part or all of the overhead raw C₄ fraction of the de-butaniser (I) or a part or all of the overhead C₅ fraction of the de-pentaniser (I) or imported olefinic C₄+ hydrocarbons or any mixture of the above to the selective hydrogenation unit (II),
g) sending the cracking reactor (II) outlet to the re-run column to produce a C₆+ bottom stream and a C₁-C₅ overhead, sending said overhead to the de-propaniser (II) to produce a C₁-C₃ overhead and a C₄+ bottom stream recycled in whole or in part to the selective hydrogenation unit (II), optionally withdrawing a part of said C₄+ bottom stream,
h) sending the C₁-C₃ overhead of the de-propaniser (II) to the de-ethaniser (II) to produce
a bottom C₃ stream optionally sent to the MAPD converter (II) to produce propane and propylene stream, optionally sent to the C₃ splitter (II) to produce a concentrated propylene stream as overhead and a propane rich bottom product,
an overhead stream optionally sent to a de-methaniser (II) to produce an overhead fuel gas and a C₂ bottom stream optionally sent to an acetylene converter.

4. Method according to claim 2 for debottlenecking an existing steam cracker unit of which the operation is modified from high severity to low severity operation, having a cracking zone and a fractionation zone, said fractionation zone comprising a gasoline stripper, then a fractionation configuration with first a de-ethaniser (I) (front-end de-ethaniser), followed by a de-methaniser (I) and followed by a de-propaniser (I) and a de-butaniser (I), in which, said de-ethaniser (I) is producing,
an overhead stream sent to a front-end acetylene converter (I) and then to a de-methaniser (I), said de-methaniser (I) producing a fuel gas overhead product and a C₂ bottom product that is sent to a C₂ splitter (I) to separate ethylene and ethane,
a bottoms stream comprising C₃+ sent to the de-propaniser (I),
said de-propaniser (I) receiving product from the bottom of the de-ethaniser (I) and optionally product from the bottom of the gasoline stripper (I), said de-propaniser (I) producing,
an overhead stream sent to a MAPD removing unit (I) to produce propane and propylene,
a bottoms stream comprising C₄+ sent to the de-butaniser (I) to produce an overhead raw C₄ fraction and a bottom C₅+ fraction, optionally said C₅+ fraction is subsequently sent to a de-pentaniser (I) to produce an overhead C₅ fraction and a bottom C₆+ fraction,
wherein said debottlenecking method comprises the steps of:
a) adding a selective hydrogenation unit (II);
b) adding a cracking reactor (II) comprising a catalyst selective towards light olefins in the outlet,
c) adding a re-run column, a de-propaniser (II), a de-ethaniser (II), optionally a de-methaniser (II), optionally a MAPD conversion unit (II) and optionally a C₃ splitter (II) to separate propane and propylene,
d) sending a part or all of the bottoms stream of the gasoline stripper (I) to the selective hydrogenation unit (II) and subsequently to the cracking reactor (II) at conditions effective to produce an outlet with an olefin content of lower molecular weight than that of the inlet,
e) sending a part of the bottoms stream of the de-ethaniser (I) to the de-propaniser (II), such as, not to overload the de-propaniser (I)
f) optionally sending a part or all of the overhead raw C₄ fraction of the de-butaniser (I) or a part or all of the overhead C₅ fraction of the de-pentaniser (I) or imported olefinic C₄+ hydrocarbons or any mixture of the above to the selective hydrogenation unit (II),
g) sending the cracking reactor (II) outlet to the re-run column to produce a C₆+ bottom stream and a C₁-C₅ overhead, sending said overhead to the de-propaniser (II) to produce a C₁-C₃ overhead and a C₄+ bottom stream recycled in whole or in part to the selective hydrogenation unit (II), optionally withdrawing a part of said C₄+ bottom stream,
h) sending the C₁-C₃ overhead of the de-propaniser (II) to the de-ethaniser (II) to produce
a bottom C₃ stream optionally sent to the MAPD converter (II) to produce propane and propylene stream, optionally sent to the C₃ splitter (II) to produce a concentrated propylene stream as overhead and a propane rich bottom product,
an overhead stream optionally sent to a de-methaniser (II) to produce an overhead fuel gas and a C₂ bottom stream optionally sent to an acetylene converter.

5. Method according to claim 3 wherein the MAPD removal unit (I) is a catalytic gas phase or liquid phase reactor that converts the MAPD (methyl acetylene and propadiene) selectively in mainly propylene.

6. Method according to claim 3 wherein the MAPD removal unit (I) consists in:
a MAPD distillation column (I), fed with the overhead of the de-propaniser (I) and producing an overhead having substantially C₃ hydrocarbons and a bottom product, enriched in MAPD and C₄ hydrocarbons,
a MAPD converter (I) receiving the overhead of the MAPD distillation column (I) and consisting in a catalytic gas phase or liquid phase reactor that converts the MAPD (methyl acetylene and propadiene) selectively in mainly propylene,
and sending a part or all of the bottom product of the MAPD distillation column (I) to the de-propaniser (II).

7. Method according to claim 3 wherein the MAPD removal unit (I) consists in a catalytic MAPD distillation column (I) and optionally a MAPD converter (I),
said catalytic MAPD distillation column (I) is fed with the overhead of the de-propaniser (I) and comprises a selective hydrogenation catalyst placed inside a distillation column, converting acetylenic and dienic hydrocarbons selectively into the corresponding olefins and producing an overhead product, having substantially C₃ hydrocarbons and a bottom product, having substantially C₄ hydrocarbons.
Optionally sending the overhead of the catalytic MAPD distillation column (I) to a MAPD converter (I) to selectively convert the remaining MAPD in propylene
Sending a part or all of the bottom product of the catalytic MAPD distillation column (I) to the de-propaniser (II) or to the selective hydrogenation unit (II).

8. Method according to claim 3 wherein the de-propaniser (I) is a catalytic de-propaniser (I), said catalytic de-propaniser (I) is fed with the bottom product of the de-ethaniser (I) and optionally product from the bottom of the gasoline stripper (I), and comprises a selective hydrogenation catalyst placed inside a distillation column, converting acetylenic and dienic hydrocarbons selectively into the corresponding olefins and producing an overhead product, having substantially C₃ hydrocarbons and a bottom product, having substantially C₄+ hydrocarbons.

9. Method according to claim 1 wherein the catalyst (A1) in the cracking reactor (II) (OCP reactor) is selected among the crystalline silicates and the phosphorus modified zeolites.

10. Method according to claim 9 wherein the crystalline silicates are selected among the crystalline silicates having a ratio Si/Al of at least about 100 and the dealuminated crystalline silicates.

11. Method according to claim 10 wherein the crystalline silicate having a ratio Si/Al of at least about 100 and the dealuminated crystalline silicate are selected among the MFI, MEL, FER, MTT, MWW, TON, EUO, MFS and ZSM-48 family of microporous materials consisting of silicon, aluminium, boron and oxygen.

12. Method according to claim 11 wherein the crystalline silicate having a ratio Si/Al of at least about 100 is selected among the MFI and the MEL.

13. Method according to claim 10 wherein the Si/Al ratio of the crystalline silicate ranges from 100 to 1000.

14. Method according to claim 10 wherein the crystalline silicate having a ratio Si/Al of at least about 100 or the dealuminated crystalline silicate is steamed to remove aluminium from the crystalline silicate framework.

15. Method according to claim 14 wherein, further to the steaming, aluminium is extracted from the catalyst by contacting the catalyst with a complexing agent for aluminium to remove from pores of the framework alumina deposited therein during the steaming step thereby to increase the silicon/aluminium atomic ratio of the catalyst.

16. Method according to claim 1 wherein the temperature of the OCP reactor (cracking reactor (II)) ranges from 540° C. to 590° C.
